(19) United States
(12) Patent Application Publication
(10) Pub. No.: US 2024/0238224 A1
(43) Pub. Date: Jul. 18, 2024

(54) COMPOSITION FOR LOCAL ANAESTHESIA AND USES THEREOF

(71) Applicant: Michael D. Smilanich, Medina, MN (US)

(72) Inventor: Michael D. Smilanich, Medina, MN (US)

(21) Appl. No.: 18/410,693

(22) Filed: Jan. 11, 2024

Related U.S. Application Data
(60) Provisional application No. 63/438,892, filed on Jan. 13, 2023.

(51) Int. Cl. A61K 31/167; A61K 31/137; A61P 23/02

(52) U.S. Cl. CPC A61K 31/167; A61K 31/137; A61P 23/02

(57) ABSTRACT

The disclosure relates to a pharmaceutical composition comprising a combination of tetracaine and bupivacaine, and various dosage forms of the same. The pharmaceutical composition can comprise 3.6% (w/v) or more tetracaine or pharmaceutically acceptable salt thereof, 0.1% (w/v) or more bupivacaine or pharmaceutically acceptable salt thereof, a vasoconstrictor, and a pharmaceutically acceptable carrier. Also described is a spray device, comprising the pharmaceutical composition, a reservoir, and an atomizer. The disclosure further relates to a method of providing local anesthesia to a site of a patient and a method of treating a method of treating a patient without use of an injected local anesthetic.

### CLAIM OF PRIORITY

This patent application claims the benefit of priority to U.S. Application Ser. No. 63/438,892, filed Jan. 13, 2023, which is incorporated by reference herein in its entirety.

### FIELD OF THE INVENTION

The disclosure relates to methods, compositions, and devices for inducing anesthesia.

### BACKGROUND

Pain management is a central pillar of medical treatment. Local anesthetics are commonly relied upon for pain management during dental, dermatologic, and minor surgical procedures. Existing pain management techniques can involve injections or use of needles. Some available anesthetic agents are unsuitable for application to mucosal tissue, wounds, or the oral cavity.

Tetracaine is a local anesthetic having the chemical name 2-(dimethylamino)ethyl 4-(butylamino)benzoate. A nasal spray composition, Kovanaze, containing tetracaine hydrochloride at a concentration of 3% (w/v) at a dose of 12-18 mg has been indicated for regional anesthesia when performing a restorative procedure on Teeth 4-13 and A-J in adults and children who weigh 40 kg or more. See, FDA Labelling for Kovanaze Nasal Spray. However, Kovanaze has several significant limitations: it is effective only on the teeth of the maxillary arch numbered 4-13 and has reduced efficacy for teeth numbered 4 and 13; it has been associated with significant adverse events; and its dosing requires multiple applications spaced 4-10 minutes apart. See, FDA Labelling for Kovanaze Nasal Spray. Other common local anesthetics include lidocaine, which is 2-(diethylamino)-N-(2,6-dimethylphenyl)acetamide, and benzocaine, which is Ethyl 4-aminobenzoate. However, such agents can be toxic and can be inappropriate for use on mocusal tissue, wounds, and the like. For some purposes where powerful local anesthesia is indicated, use of such agents may be foreclosed or require skilled use of needle injection.

There is a need for new pain management tools that are effective for providing local anesthesia for various medical and dental procedures without imparting unacceptable levels of toxicity. There is also a need for new pain management tools that do not involve needles or injections.

### SUMMARY OF THE DISCLOSURE

The disclosure herein provides compositions, methods, and other treatments useful for inducing local anesthesia in a site of a patient in need thereof.

The present disclosure provides a pharmaceutical composition comprising tetracaine, or a pharmaceutically acceptable salt thereof; bupivacaine, or a pharmaceutically acceptable salt thereof; a vasoconstrictor; and a pharmaceutically acceptable carrier. The pharmaceutical composition can have 3.6% (w/v) or more tetracaine, or a pharmaceutically acceptable salt thereof; and 0.1% (w/v) or more bupivacaine.

The present disclosure provides a spray device comprising a pharmaceutical composition, a reservoir, and an atomizer. The pharmaceutical composition comprises tetracaine, or a pharmaceutically acceptable salt thereof; bupivacaine, or a pharmaceutically acceptable salt thereof; a vasoconstrictor; and a pharmaceutically acceptable carrier.

The present disclosure also provides a method of providing local anesthesia to a site of a patient, comprising topically administering to the patient the pharmaceutical composition, or applying the spray device. The pharmaceutical composition comprises tetracaine, or a pharmaceutically acceptable salt thereof; bupivacaine, or a pharmaceutically acceptable salt thereof; a vasoconstrictor; and a pharmaceutically acceptable carrier.

Advantages, some of which are unexpected, are achieved by various aspects of the present disclosure. For example, the present disclosure describes various pharmaceutical compositions that are capable of inducing anesthesia where other topical anesthetics have failed. For example, in various aspects, the present disclosure describes a composition that achieves profound pulpal anesthesia for teeth numbered 1-3 and 14-16 when used topically.

In various aspects, the disclosure surprisingly and advantageously provides dosage forms of the pharmaceutical composition that achieves the desired anesthesia by using only a single spray dose. Additionally, the disclosure achieves a sprayable formulation that eliminates adverse events of prolonged congestion, rhinalgia, prolonged sinusitis, and epistaxis. It is especially surprisingly that, in various aspects, the pharmaceutical composition has the advantage of reduced side effects and also the advantage increased efficacy. As a yet further advantage, the innovative pharmaceutical composition, and new suitable modes of administration, surprisingly render a viscous formulation of approximately 4500 cP at 25° C. to be particularly advantageous for applying to oral tissue of a subject.

### BRIEF DESCRIPTION OF THE DRAWINGS

FIG. 1 provides an illustration summarizing the universal numbering maxillary teeth and shows that the molars (1-3 and 14-16) are enervated by the posterior superior alveolar as indicated by the solid line. In about one third of patients, the second premolar (4 and 13) are also enervated by the PSA, as indicated by the dashed line.

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Although any methods and materials similar or equivalent to those described herein may be useful in the practice or testing of the present invention, certain methods and materials are described below. Specific terminology of particular importance to the description of the present invention is defined below.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that section.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

A “pharmaceutical composition” refers to a composition suitable for administration to a subject (e.g., mammal) to achieve a therapeutic result. Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectal, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration. In various aspects, the pharmaceutical composition can be a topical composition, or a composition that is not formulated, or not suitable, for administration by injection.

A “topical pharmaceutical composition” refers to a composition suitable for administration to a subject (e.g., mammal) by topical means, which can include one or more of buccal, sublingual, dermal, transmucosal, intranasal, ocular, or by placing the pharmaceutical composition on an oral surface such as the tongue, gums, palate, or teeth.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions. The carrier can be a liquid, in which an active therapeutic agent is formulated. The carrier or excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety. As further examples, the pharmaceutically acceptable carrier can include water, a water-alcohol mixture, an aqueous buffer solution, or an aqueous solution of sodium chloride. In various aspects, the carrier is saline; while in other aspects, the carrier is not saline. In various aspects, the buffer solution can include sodium hydroxide. For example, the composition can comprise 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% of a base solution solution, such as sodium hydroxide at a concentration of 0.1N.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof.

The compositions can be administered by a variety of dosage forms as known in the art. Any biologically-acceptable dosage form known to persons of ordinary skill in the art, and combinations thereof, are contemplated. Examples of such dosage forms include, without limitation, aerosols, nasal sprays, topical sprays, a wound care spray or sprays for the skin, liquids, solutions, suspensions, emulsions, lozenges, chewable lozenges, beads, powders, gum, foam, granules, particles, microparticles, dispersible granules, cachets, douches, suppositories, creams, gels, pastes, topicals, inhalants, aerosol inhalants, patches, particle inhalants, implants, depot implants, ingestibles, injectables (including subcutaneous, intramuscular, intravenous, and intradermal), non-injectables, infusions, and combinations thereof. In various aspects, the dosage form is a non-injectable dosage form. In various aspects, the dosage form is an aerosols, nasal spray, or topical spray, and it can be contained within a drug delivery device for providing the same. In various aspects, the drug delivery device is a metered dose delivery device. Such devices can be configured to provide a particular dose; for example, a dose of less than 200 microliters, a dose of 50-150 microliters, a dose about 100 microliters, a dose of less than 12 mg of tetracaine, a dose of less than 5 mg of tetracaine, a dose of 3-6 mg of tetracaine, or a dose of about 4 mg of tetracaine.

The composition can, in various aspects, further include lactose, dextrose, saccharose, cellulose, methylcellulose, starch, calcium phosphate, olive oil or ethyl oleate for soft capsules and water or vegetable oil for suspensions or emulsions; lubricating agents such as silica, talc, stearic acid, magnesium or calcium stearate and/or polyethylene glycols; thickening agents, which can be gelling agents, such as starches, arabic gums, gelatin, or cross-linking polymers suitable for biologic administration. In further examples, the composition can include gum tragacanth, sodium alginate, cellulose, methylcellulose, carboxymethylcellulose, hydroxyethylcellulose, hydroxyalkylcellulose, hydroxypropylcellulose, smart hydrogenl, polyvinylpyrrolidone, starch, agar, pectin, carboxymethylcellulose, polyvinyl alcohol, alginic acid, other alginates, sodium starch glycolate. The composition can also include dyestuff, sweeteners, flavors, wetting agents such as lecithin, polysorbates or laurylsulphates; and other therapeutically acceptable accessory ingredients, such as humectants, preservatives, buffers and antioxidants. The composition can also include mineral acids, mineral bases, organic acids, or organic bases, which are suitable for biologic administration for adjusting salt forms of various ingredients, or for adjusting pH of the composition. The composition can further include isotonic agents, for example, sugars, polyalcohols such as mannitol, sorbitol, or sodium chloride in the composition. The pharmaceutical compositions can include one or more suitable production aids or excipients including fillers, binders, disintegrants, lubricants, diluents, flow agents, buffering agents, moistening agents, and preservatives. In various examples, the preservative includes benzyl alcohol. The concentration of the preservative, e.g., benzyl alcohol, can be about 0.01% to about 5% (w/v), about 0.01% to about 1% (w/v), about 0.1% to about 1% (w/v), about 0.01% (w/v), about 0.05% (w/v), about 0.10% (w/v), about 0.25% (w/v), about 0.50% (w/v), about 0.60% (w/v), about 0.70% (w/v), about 0.80% (w/v), about 0.90% (w/v), or about 1.0% (w/v), or any range between such values.

A “dosage unit form,” as used herein, refers to physically discrete units suited as unitary dosages for the mammalian subjects to be treated; each unit containing a predetermined quantity of active compound calculated to produce the desired therapeutic effect in association with the pharmaceutical carrier. For example, in various aspects, the pharmaceutical composition can be configured as a dosage unit form corresponding to a dose of less than 200 microliters, a dose of 50-150 microliters, a dose about 100 microliters, a dose of less than 12 mg of tetracaine, a dose of less than 5 mg of tetracaine, a dose of 3-6 mg of tetracaine, or a dose of about 4 mg of tetracaine, which can be in the form of an aqueous solution, a semi-solid, a paste, an ointment, a gel, an aerosol, a nasal spray, a topical spray, or a wound care spray. The dosage unit can correspond to a single application of a spray, or a single application of a pre-determined amount of a liquid, semi-solid, or solid composition applied to the patient.

In therapeutic use for treatment of conditions in mammals (e.g., humans) for which the appropriate pharmaceutical composition is effective, or for the induction of local anesthesia at a site of a patient, the active ingredients of the presently described pharmaceutical composition can be administered in an effective amount.

As used herein, the term “administration” or “administering” refers to any means of providing a therapeutic agent (e.g., a pharmaceutical composition for providing anesthesia) to a subject for the purpose of treating the subject. In various aspects, administration can be provided without needles, or without injection, or both. Administration can can involve a single administration of a dose, or it can involve administration of multiple doses, such as twice or three times. In various aspects, local anesthesia of a desired site of a patient can be achieved in a single dose.

The administration can be local, where the composition is administered directly, close to, in the locality, near, at, about, proximate to, or in the vicinity of, the site(s) for which anesthesia is desired, or the site for which pain management is indicated. Local administration can be administration to, for example, a tooth, tissue, organ, or region thereof. Local administration can be non-injected administration. Local administration can be topical administration. Administration can be topical with a local effect, composition is applied directly where its action is desired. Administration can be applied to a nerve implicated in pain management of the site for which anesthesia is desired.

The administration can be topically, where the composition is administered by contacting the pharmaceutical composition directly, close to, in the locality, near, at, about, proximate to, or in the vicinity of, a tissue, nerve, tooth, or region that serves to induce local anesthesia at the desired site. The contacting can contact a surface of the tissue, nerve, tooth, or region, which includes an oral surface, surface of the sinuses, nasal surface, a skin surface, a tooth, gums, hard palate, soft palate, and the like. Topical administration can be achieved by for example: placing, rubbing, or spraying, the composition onto the surface being treated. For example, the topical composition can be placed along the upper portion of a patient's gum near the top part of the one or more maxillary tooth, or maxillary tissue, for which anesthesia is desired. As another example, the topical composition can be placed upon the infraorbital foramen.

In various aspects, the pharmaceutical composition is provided to the patient in a therapeutically effective amount. The term “therapeutically effective amount” as used herein, refers to that amount of the pharmaceutical composition that elicits a biological or medicinal response in a tissue system, animal or human, that is being sought by a researcher, veterinarian, medical doctor, dentist, or other clinician, which includes achieving anesthesia. The response sought can be the prevention of pain or sensation. In some examples, the therapeutically effective amount is that which can sufficiently achieve anesthesia so as to justify performing a given medical treatment with a reasonable benefit/risk ratio with respect to patient comfort. However, it is to be understood that the usage of the compositions described herein can be decided by the clinician within the scope of sound medical judgment. The specific therapeutically-effective dose and method of administration for any particular patient can depend upon a variety of factors, including the anesthesia being sought or condition being treated; the age, body weight, general health, gender and diet of the patient: the time of administration, route of administration, and the duration of the treatment.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. In various aspects, the pharmaceutically acceptable salt is a hydrochloride.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, the disclosure of which is hereby incorporated by reference.

In various aspects, one or more active ingredients can be in a prodrug form. The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for anesthetic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

As used herein, the term “anesthesia” refers to a loss of sensation in a given site (e.g., a tissue or tooth) of a patient. The anesthesia can deeply penetrate tissue sufficiently to target nerves, nerve branches, or tissue at a depth of 3 mm or greater. Such anesthesia thus refers to anesthetizing more than merely the outer 2 mm surface of the treated tissue or region. In various aspects, the extent of anesthesia can be qualified by use of electric pulp testing (EPT) stimulation. The term “pulpal anesthesia” can be used to describe a level of anesthesia such that stimulation of a patient's tooth with EPT stimulation at a reading of 65 or above (on a standard 0-80 EPT scale) results in no pain sensation. The term “profound anesthesia” can be used to describe achieving anesthesia within 30 minutes after administration and continuing for at least 20 additional minutes, and wherein the anesthesia corresponds to a lack of sensation at the site when it is stimulated with an electric pulp tester set to 80 on a standard EPT scale of 0 to 80. Use of ENDO-ICE™ corresponds to an electric pulp tester (EPT) set to at least 65 on a standard EPT scale of 0 to 80. As used herein, the term “local anesthetic” and “local anesthesia” refers to an anesthetic or anesthesia that achieves anesthesia of a given site of a patient without anesthetizing the entire patient.

As used herein, the term “anesthetizing” refers to administering an anesthetic composition for the purpose of preventing sensation, preventing pain, or both. In various aspects, anesthetizing refers to inducing local anesthesia.

As used herein, the term “intranasal formulation” and the like refers to a formulation that is suitable, or configured, for delivery to the nasal cavity or nasal sinuses.

As used herein, the term “mucosal” tissue can refer to oral or non-oral tissue. Examples of oral mucosal tissue include mucosal tissue of the throat, mouth, and nose. Examples of non-oral mucosal tissue include ear, eye, rectal, or urogenital tissue. Further examples of non-oral mucosal tissue include vaginal and urethral mucosa.

As used herein, the term “skin” refers to non-oral skin.

As used herein, the term “maxillary” refers to the teeth, tissue, and the like, of the upper jaw.

### Pharmaceutical Composition, Devices, and Uses

The present disclosure provides a pharmaceutical composition, which contains a combination of tetracaine, bupivacaine, a vasoconstrictor, and a pharmaceutically acceptable carrier. As such, this disclosure relates generally to use of a combination of multiple active ingredients, particularly a combination of tetracaine, bupivacaine, and a vasoconstrictor.

Combination therapies using multiple active agents to achieve a particular result, e.g., local anesthesia without use of a needle, can involve complex interactions and results that are not reasonably predictable without clinical testing. The effects of any single drug are related to its absorption, distribution, and elimination, its active effect, efficacy, and side effect profile. When two drugs are introduced to a patient, each drug can affect and, thus, alter the effects of the other. For instance, one drug may inhibit, activate or induce a biological state that is implicated with the other drug (Guidance for Industry. In vivo drug metabolism/drug interaction studies-study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999). Thus, when two drugs are administered to treat the same condition, it can be unpredictable whether each will complement, have no effect on, or interfere with, the therapeutic activity of the other in a human subject. Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it can be unpredictable what change will occur in the negative side profile of each drug. It is difficult and sometimes not feasible to estimate the effects of the interaction between two drugs (Guidance for Industry, 1999). Combination therapies involving three or more drugs can be even more complex.

Tetracaine is (2-(dimethylamino)ethyl 4-(butylamino)benzoate) or a salt thereof. An example salt of tetracaine is tetracaine HCl. The concentration of the tetracaine, or salt thereof, in the pharmaceutical composition can be about 3.6% to about 10% (w/v), about 3.6% to about 7% (w/v), about 3.6% to about 6% (w/v), about 3.6% to about 5% (w/v), about 3.9% to about 10% (w/v), about 3.9% to about 7% (w/v), about 3.9% to about 6% (w/v), about 3.9% to about 5% (w/v), about 4% to about 10% (w/v), about 4% to about 7% (w/v), about 4% to about 6% (w/v), or about 4% to about 5% (w/v). The tetracaine, or salt thereof, can be at a concentration of at least or about 3.6% (w/v), 3.7% (w/v), 3.8% (w/v), 3.9% (w/v), 4.0% (w/v), 4.1% (w/v), 4.2% (w/v), 4.3% (w/v), 4.4% (w/v), 4.5% (w/v), 4.6% (w/v), 4.7% (w/v), 4.8% (w/v), 4.9% (w/v), or about 5% (w/v), or any range between such values. In various aspects, the concentration of the tetracaine salt is configured to provide free tetracaine at a concentration of at least or about 3.6% (w/v), 3.7% (w/v), 3.8% (w/v), 3.9% (w/v), or 4% (w/v). In various aspects, the pharmaceutical composition has about 4.0% (w/v) or more tetracaine, or a pharmaceutically acceptable salt thereof.

Bupivacaine is 1-butyl-N-(2,6-dimethylphenyl)piperidine-2-carboxamide or a salt thereof. An example salt of bupivacaine is bupivacaine HCl. The concentration of bupivacaine, or salt thereof, in the pharmaceutical composition can be about 0.1% to about 5% (w/v), about 0.1% to about 2% (w/v), about 0.1% to about 1% (w/v), about 0.25% to about 5% (w/v), about 0.25% to about 2% (w/v), about 0.25% to about 1% (w/v), about 0.5% to about 5% (w/v), about 0.5% to about 2% (w/v), or about 0.5% to about 1% (w/v), The bupivacaine, or salt thereof, can be at a concentration of at least or about 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), or 1.0% (w/v), or any range between such values. In various aspects, the concentration of the bupivacaine salt is configured to provide free bupivacaine at a concentration of at least or about 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), or 1.0% (w/v). In various aspects, the pharmaceutical composition has about 0.25% (w/v) of bupivacaine, or a salt thereof. In various aspects, the pharmaceutical composition has about 0.50% (w/v) of bupivacaine, or a salt thereof.

In various aspects, the pharmaceutical composition can include: 3.6% (w/v) or more tetracaine, or a pharmaceutically acceptable salt thereof; 0.1% (w/v) or more bupivacaine, or a pharmaceutically acceptable salt thereof; a vasoconstrictor; and a pharmaceutically acceptable carrier.

In various aspects, the vasoconstrictor can be phenylephrine or a non-phenylephrine vasoconstrictor, or a pharmaceutically acceptable salt thereof. Examples of a non-phenylephrine vasoconstrictor include oxymetazoline, xylometazoline, guanfacine, clonidine, meperidine, capsaicin, icilin, and pharmaceutically acceptable salts thereof. In various aspects, the vasoconstrictor is other than oxymetazoline or oxymetazoline derivatives. The concentration of the vasoconstrictor can be about 0.01% to about 5% (w/v), about 0.01% to about 1% (w/v), about 0.1% to about 1% (w/v), about 0.01% (w/v), about 0.05% (w/v), about 0.10% (w/v), about 0.25% (w/v), about 0.50% (w/v), about 0.60% (w/v), about 0.70% (w/v), about 0.80% (w/v), about 0.90% (w/v), or about 1.0% (w/v), or any range between such values. In various aspects, the vasoconstrictor is phenylephrine.

In various aspects, the ratio of tetracaine:bupivacaine can be about 8:1. In various further aspects, the ratio of tetracaine:bupivacaine can be in a range of about 2-20:1, 4-12:1, 6-10:1, or about 7-9:1. For example, the pharmaceutical composition can have about 4.0-6.0% (w/v) tetracaine, or a pharmaceutically acceptable salt thereof; and about 0.25-1.0% (w/v) bupivacaine, or a pharmaceutically acceptable salt thereof. As another example, the pharmaceutical composition can have about 4.0% (w/v) tetracaine, or a pharmaceutically acceptable salt thereof; and about 0.5% (w/v) bupivacaine, or a pharmaceutically acceptable salt thereof.

The pharmaceutical composition can be formulated as topical composition, including a sprayable composition. As used herein, the term “topical” composition can be understood, per context, to include sprayable compositions including an intranasal spray or oral spray. The composition can be formulated as a topical composition for oral tissue (e.g., gums) or non-oral tissue (e.g., skin). The oral tissue can be one or more of gums, teeth, soft palate, hard palate, tongue, tonsils, mucosal tissue or non-mucosal tissue. The non-oral tissue can be non-oral mucosal tissue, an epidermal tissue, a dermal tissue, or a sub-dermal tissue. Sub-dermal tissue can include hypodermis and tissues underlying the hypodermis. The topical composition can be formulated for application to a wound, which can be an open wound, a closed wound, a sutured wound, a laceration, a burn, a wound site intended for suturing, an implant site, a biopsy site or a surgical site. In various aspects, the pharmaceutical composition can be formulated as an aqueous composition.

The pharmaceutical composition can be formulated as a topical gel, paste, semi-solid, or viscous liquid. The composition can include a thickening agent. The thickening agent can be a biologically acceptable cross-linking ingredient, or it may be a non-crosslinking ingredient that increases viscosity. The thickening agent can provide a gel formulation. In various aspects, the thickening agent can comprise, for example, modified celluloses (e.g., hydroxypropyl cellulose and hydroxyethyl cellulose), carbopol homopolymers and copolymers, solvents such as diglycol monoethyl ether, alkylene glycols (e.g., propylene glycol), dimethyl sosorbide, alcohols (e.g., isopropyl alcohol and ethanol), isopropyl myristate, ethyl acetate, C—C alkylbenzoates, mineral oil, squalane, cyclomethicone, capric/caprylic triglycerides, or combinations thereof. The amount of thickening agent can be adjusted up to the desired level of viscosity. In various aspects, the composition has a viscosity of 2000-10,000 cP, 3000-10000 cP, 3000-7000 cP, 3000-6000 cP, 4000-6000 cP, 4000-5000 cP, or about, at least, or up to, 3500 cP, 4000 cP, 4100 cP, 4200 cP, 4300 cP, 4400 cP, 4500 cP, 4600 cP, 4700 cP, 4800 cP, 4900 cP, 5000 cP, or 5500 cP. For example, the viscosity can have a viscosity corresponding to honey or molasses. In various other aspects, the composition has a viscosity sufficiently low as to permit atomization, for example, using a MAD Nasal™ Spray atomization device available from Teleflex® (Wayne, PA). In various other aspects, the composition has a viscosity that is greater than suitable for atomization, for example, using a MAD Nasal™ Spray atomization device available from Teleflex® (Wayne, PA). For example, in various aspects, the composition has at least, or about, 0.5%, 0.1%, 0.5%, 1%, 2%, 3%, 4%, 5%, 6%, 7%, 8%, 9%, 10%, 11%, 12%, 13%, 14%, 15%, or 20% by weight/volume (w/v).

There is need for a topical anesthetic that provides anesthesia of the soft tissues, including the soft and hard palate, in dentistry. There is a need for such anesthetics that provide pulpal anesthesia, and there is a need for such anesthetics that provide profound anesthesia. Such anesthetics are useful prior to performing a maxillary palatal injection, deep scaling and root planning, tissue biopsy, frenectomy, crown and bridge procedures involving retraction, suture placement, and implant uncovering procedures. A conventional approach is to use benzocaine-based anesthetics, but such approach can be unsafe and ineffective. See FDA Warning Letter. In contrast, the present disclosure provides a solution to a problem that was unsolved prior to the filing date of the present application. Specifically, the pharmaceutical composition can be applied as a needle-free topical directly onto the soft tissues to be anesthetized. The pharmaceutical composition can be modified with a thickening agent to increase its viscosity. A viscosity of 4500 cP at 25° C. can be advantageous for applying the pharmaceutical composition in this manner.

For example, the disclosure provides a topical gel composition, comprising about 4% (w/v) tetracaine, or a pharmaceutically acceptable salt thereof; about 0.5% (w/v) bupivacaine, or a pharmaceutically acceptable salt thereof; phenylephrine; a pharmaceutically acceptable carrier; and a thickening agent at amount sufficient to provide a viscosity of about 4000-5000 cP at 25° C.

The pharmaceutical composition can be formulated as a sprayable composition, or as an aerosol. In addition to the tetracaine, bupivacaine, vasoconstrictor, and pharmaceutically acceptable carrier, the sprayable pharmaceutical composition can also include eucalyptol, menthol, camphor, or any combination thereof. As discussed in further detail herein, use of eucalyptol, menthol, and camphor in the spray has the surprising advantage that a combination of tetracaine and bupivacaine results in reduces adverse effects. For example, in various aspects, the pharmaceutical composition results in reduced adverse effects, such as one or more of congestion, rhinalgia, sinusitis, and epistaxis, in a patient relative to an otherwise identical pharmaceutical composition that lacks the eucalyptol, menthol, and camphor administered in the same manner. The mode of administration can be intranasal administration. As a further example, in various aspects, the patient is free from adverse events of congestion, rhinalgia, sinusitis, and epistaxis for 7 days following administration of the pharmaceutical composition.

The composition can include a propellant, so as to provide a foam formulation or so as to facilitate spray administration. Examples of gaseous propellants include, but are not limited to, hydrofluoroalkanes (HFAs) such as 1,1,1,2-tetrafluoroethane (HFA 134a) and 1,1,1,2,3,3,3-heptafluoropropane (HFA227).

The disclosure provides a sprayable composition, which can be an intranasal spray, comprising: about 4% (w/v) tetracaine, or a pharmaceutically acceptable salt thereof; about 0.5% (w/v) bupivacaine, or a pharmaceutically acceptable salt thereof; phenylephrine; a pharmaceutically acceptable carrier; and eucalyptol, menthol, and camphor.

The disclosure provides a wound spray, oral spray, or skin spray, which can be an intranasal spray, comprising: about 4% (w/v) tetracaine, or a pharmaceutically acceptable salt thereof; about 0.5% (w/v) bupivacaine, or a pharmaceutically acceptable salt thereof; and phenylephrine. The disclosure also provides a spray device, or kit, for delivering the pharmaceutical composition described herein to a patient in the form as a spray. The spray device or kit can include a reservoir and an atomizer. The reservoir can contain the pharmaceutical composition or can be suitable for containing the pharmaceutical composition. The atomizer can be a pump sprayer or a positive pressure nebulizer. The device can take the form of a squeeze bottle, syringe, or pneumatically pressurized device. The kit can include one or more of the pharmaceutical composition, reservoir, and atomizer, but include instructions for using each of the pharmaceutical composition, reservoir, and atomizer together to administer a dose of the pharmaceutical composition as a spray. In various aspects, the spray device is a syringe that is pre-filled with the pharmaceutical composition and outfitted with an atomizer at the tip of the syringe. The various aspects, the spray device is a MAD Nasal™ Spray atomization device, which is available from Teleflex® (Wayne, PA), that is modified to contain the pharmaceutical composition described herein. In various aspects, the spray device can utilize a spray nozzle that maintains sterility of the composition within the spray device. For example, the spray device can utilize an Aptar APF or Aptar APF Plus (Crystal Lake, Illinois). In further examples, the spray device can utilize a glass reservoir.

The spray device, or kit, can be configured to provide a pre-determined dose of the pharmaceutical composition. For example, the spray device, or kit, can be configured to provide an aerosolized dose of less than 200 microliters of the pharmaceutical composition. The dose can be about 20-180, 30-170, 40-160, 50-150, 60-140, 70-130, 80-120, or 90-110 microliters of the pharmaceutical composition. In various aspects, the dose is less than, or about, 180, 170, 160, 150, 140, 130, 120, 110, 100, 90, 80, 70, 60, or 50 microliters. For example, the dose can be 100 microliters. The spray device, or kit, can be configured to provide a dose of less than 18 mg of tetracaine, a dose of less than 12 mg of tetracaine, a dose of less than 6 mg of tetracaine, or a dose of less than 5 mg of tetracaine. The dose can be 1-18, 1-17, 1-16, 1-15, 1-14, 1-13, 1-12 mg, 2-11 mg, 1-10 mg, 1-9 mg, 2-8 mg, 2-7 mg, 2-6 mg, or 2-5 mg. The dose can be about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, or about 12 mg, or at an intermediate value between any combination of such amounts. In various aspects, such doses are in the form of a single dose (e.g., as a single spray, or a single placement of material). The dose can be about 4 mg of tetracaine. The spray device can be configured to contain multiple doses or a single metered dose. The spray device can be configured to contain 10 to 150 metered doses. The spray device can be configured to contain 12,000 microlitters of composition. The spray device can be configured to contain about 120 metered doses. The spray device can be configured so that five metered dosages represents a suitable maximum dosage per patient per day.

The spray device can be configured as an intranasal spray device for delivering the aerosol to nasal and oropharyngeal mucous membranes, or as a non-nasal spray device, such as a device configured to spray onto a wound, into the mouth, or onto the skin. In various aspects, the spray device takes the form of a syringe, which is equipped with an atomizer at the tip of the syringe in lieu of a needle. The syringe includes a plunger and a reservoir. The reservoir of the syringe can contain the pharmaceutical composition and pressing on the plunger drives the pharmaceutical composition through the atomizer. In various further aspects, the kit can also take the form of a syringe but includes instructions for using the syringe to deliver a spray of the pharmaceutical composition at the desired dose; or the kit can include the pharmaceutical composition with instructions for using a syringe and atomizer to deliver a spray of the pharmaceutical composition at the desired dose.

The disclosure further provides a sprayable drug-device combination, comprising an aqueous pharmaceutical composition, a reservoir, and an atomizer, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier.

The disclosure also provides a method of providing local anesthesia at a site of a patient. The method can involve topically administering to the patient a pharmaceutical composition described herein, which can be provided by way of an spray drug delivery device described herein.

The site of the patient can be one or more tooth, tissue, nerve, or organ system. In various aspects, the site can be a tooth or an oral tissue. For example, the site can be on or more one adult tooth numbered 1-3 or 13-16, or adult tooth numbered 4 or 12 that is enervated by a posterior superior alveolar (PSA) nerve in the patient, based on universal tooth numbering. In various further examples, the tooth can be a molar, such as a first molar, second molar, third molar, or any combination thereof. The tooth can be a premolar, such as a premolar that is enervated by a posterior superior alveolar (PSA) nerve in the patient. The tooth can be a cuspid, lateral incisor, or central incisor. The site can be one or more tooth numbered 1-16, 17-32, A-J, K-T, or any combination thereof. The site can be one, two, three, four, or more maxillary teeth, or all maxillary teeth, or one, two, three, four, or more mandibular teeth, or all mandibular teeth. The site can be a maxillary region of teeth, tissue or both, or a mandibular region of teeth, tissue, or both. The maxillary region can be all maxillary teeth, two or more maxillary molars, all maxillary molars, all teeth in an upper right quadrant, all teeth in an upper left quadrant, a hard palate, a soft palate, gums, an implant, a posterior superior alveolar (PSA) nerve, or any combination thereof. The site can be one or more nerve that enervates one or more of the aforementioned teeth. The nerve can be a posterior superior alveolar (PSA) nerve. The site can be an oral tissue, such as an oral tissue enervated by a posterior superior alveolar (PSA) nerve. The site of the patient can be an oral tissue or a non-oral tissue. The oral tissue can be one or more of gums, teeth, soft palate, hard palate, tongue, tonsils, mucosal tissue or non-mucosal tissue. The non-oral tissue can be non-oral mucosal tissue, an epidermal tissue, a dermal tissue, or a sub-dermal tissue. The site can be a wound, which can be an open wound, a closed wound, a sutured wound, a laceration, a burn, a wound site intended for suturing, an implant site, a biopsy site or a surgical site. The site can be site intended for injection, or a site other than a site intended for injection.

Various modes of administration can be used to induce anesthesia using the pharmaceutical compositions described herein. In various aspects, anesthesia can be induced without use of injection or needles.

In various aspects, the pharmaceutical composition can be administered by placing it along the upper portion of a patient's gum near the top part of the one or more maxillary tooth, or maxillary tissue, for which anesthesia is desired. This mode of administration can be used, for example, to achieve anesthesia of one or more maxillary tooth. In various other aspects, the pharmaceutical composition can be administered by placing it at the patient's infraorbital foramen. This mode of administration can be used, for example, to achieve anesthesia of a maxillary region of teeth, tissue, or a combination thereof. The pharmaceutical composition can be a gel, paste, or other viscous form that is administered by placing it upon a patient tissue site. For example, the pharmaceutical composition can have a viscosity of 3500-5000 cP.

In various aspects, the pharmaceutical composition can be formulated, administered, or both, as a spray. For example, the pharmaceutical composition can be administered as a nasal spray. The nasal spray can be delivered ipsilaterally to the site for which anesthesia is desired. In various examples, the pharmaceutical composition can be formulated to be suitable for spraying, e.g., as a sprayable formulation. The sprayable formulation can further comprise eucalyptol, menthol, camphor, or any combination thereof. As discussed in further detail herein, use of eucalyptol, menthol, and camphor in the spray has the surprising advantage that a combination of tetracaine and bupivacaine results in reduces adverse effects. For example, in various aspects, the pharmaceutical composition results in reduced adverse effects, such as one or more of congestion, rhinalgia, sinusitis, and epistaxis, in a patient relative to an otherwise identical pharmaceutical composition that lacks the eucalyptol, menthol, and camphor. As a further example, in various aspects, the patient is free from adverse events of congestion, rhinalgia, sinusitis, and epistaxis for 7 days following administration of the pharmaceutical composition.

In various aspects, the pharmaceutical composition is administered in the form of a topical spray delivered directly to the site for which anesthesia is desired. The pharmaceutical composition can be configured for spraying onto an oral tissue or a non-oral tissue. For example, the pharmaceutical composition can be administered as a non-nasal spray, such as a wound spray, a spray for the mouth, or a spray for the skin. The pharmaceutical composition can be configured for spraying onto a wound, which can be an open wound, a closed wound, a sutured wound, a laceration, a burn, a wound site intended for suturing, an implant site, a biopsy site, or a surgical site. The pharmaceutical composition can be configured for spraying onto a site intended for injection, or a site other than a site intended for injection.

In various aspects, the pharmaceutical composition can achieve local anesthesia at the site of the patient without use of any injection, e.g., without injecting a local anesthetic, or without applying any needle to the patient.

The disclosure also provides a method of providing local anesthesia to an adult molar numbered 1-3 or 13-16, based on universal tooth numbering, comprising topically administering a pharmaceutical composition to a tissue proximate to the molar being treated in a patient in need thereof, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier. As another example, the disclosure provides a method of providing local anesthesia to an adult molar numbered 1-2 or 15-16, based on universal tooth numbering.

The disclosure further provides a method of providing local anesthesia to a tissue site of a patient, comprising topically administering a pharmaceutical composition directly to the tissue of the patient in need thereof, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier, wherein the tissue site is an oral tissue, a non-oral mucosal tissue, an epidermal tissue, a dermal tissue, a sub-dermal tissue, an open wound, a laceration, an implant site, a biopsy site, or a suture site.

The disclosure also provides a method of treating a patient without injecting a local anesthetic. The patient can be in need of one or more procedures that involve or require anesthetizing a site on the patient, e.g., a tissue or tooth. Anesthesia can be achieved by using the pharmaceutical composition, spray device, or method, described herein. The medical procedure can be performed upon achieving anesthesia at the site of the patient. The method can involve treating a patient without injecting a local anesthetic by: (a) topically administering the pharmaceutical composition described herein, or employing a spray device described herein, to a patient in need thereof; and (b) performing a medical procedure selected from one or more of an axillary palatal injection, deep scaling, root planning, tissue biopsy, frenectomy, crown or bridge procedure involving retraction, suture placement, an implant uncovering procedure, or a restorative procedure in a tooth numbered 1-3 or 13-16, based on universal tooth numbering, or anesthesia of adult tooth numbered 4 or 12 that is enervated by a posterior superior alveolar (PSA) nerve.

The pharmaceutical composition can be formulated, administered, or both, in an amount effective to achieve local anesthesia of the site of the patient for which anesthesia is desired. In various aspects, the pharmaceutical composition is formulated, administered, or both, as an aerosolized dose of less than 200 microliters of the pharmaceutical composition. The dose can be about 20-180, 30-170, 40-160, 50-150, 60-140, 70-130, 80-120, or 90-110 microliters of the pharmaceutical composition. In various aspects, the dose is less than, or about, 180, 170, 160, 150, 140, 130, 120, 110, 100, 90, 80, 70, 60, or 50 microliters. For example, the dose can be 100 microliters. In various aspects, the pharmaceutical composition is formulated, administered, or both, at a dose of less than 18 mg of tetracaine, a dose of less than 12 mg of tetracaine, a dose of less than 6 mg of tetracaine, or a dose of less than 5 mg of tetracaine. The dose can be 1-18, 1-17, 1-16, 1-15, 1-14, 1-13, 1-12 mg, 2-11 mg, 1-10 mg, 1-9 mg, 2-8 mg, 2-7 mg, 2-6 mg, or 2-5 mg. The dose can be about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, or about 12 mg, or at an intermediate value between any combination of such amounts. In various aspects, such doses are in the form of a single dose (e.g., as a single spray, or a single placement of material). The dose can be about 4 mg of tetracaine. In various further aspects, the pharmaceutical composition is provided in a drug delivery format, metered dosage form, or as a kit with instructions, that provides for a pre-determined amount of administered dose, e.g., a spray device configured to provide a particular dose or dose range.

The extent and sufficiency of anesthesia can be qualified by use of pulpal stimulation tools known, including ordinary tools for evaluating anesthesia that are known in the art. In various aspects, pulpal stimulation can be used, such as by using an electric pulp tester or refrigerant spray, such as ENDO-ICE™. In various aspects, the pharmaceutical composition, spray device, or method described here results in anesthesia corresponding to a lack of sensation at the site when it is stimulated with an electric pulp tester (EPT) set to at least 65 on a standard EPT scale of 0 to 80, and such anesthesia can be described herein as pulpal anesthesia. In yet further aspects, the pharmaceutical composition, spray device, or method described here results in anesthesia within 30 minutes after administration and continuing for at least 20 additional minutes, and wherein the anesthesia corresponds to a lack of sensation at the site when it is stimulated with an electric pulp tester set to 80 on a standard EPT scale of 0 to 80, and such anesthesia can be described herein as profound pulpal anesthesia. Use of ENDO-ICE™ corresponds to an electric pulp tester (EPT) set to at least 65 on a standard EPT scale of 0 to 80.

In various aspects, the local anesthesia can be suitable for performing a restorative procedure in a tooth of an adult or a child that weighs 40 kg or more. For example, a restorative procedure in a tooth numbered 1-3 or 13-16, based on universal tooth numbering, or anesthesia of adult tooth numbered 4 or 12 that is enervated by a posterior superior alveolar (PSA) nerve. As another example, a restorative procedure in a tooth numbered 1-2 or 15-16, based on universal tooth numbering. In various further aspects, the local anesthesia can be suitable for performing one or more of an axillary palatal injection, deep scaling, root planning, tissue biopsy, frenectomy, crown or bridge procedure involving retraction, suture placement, or an implant uncovering procedure. In yet further aspects, the local anesthesia is suitable for performing one or more of an axillary palatal injection, deep scaling, root planning, tissue biopsy, frenectomy, crown or bridge procedure involving retraction, suture placement, or an implant uncovering procedure.

In various aspects, the pharmaceutical composition, spray device, or method, described herein, is sufficient to achieve the desired anesthesia, is the sole means for achieving anesthesia, or both.

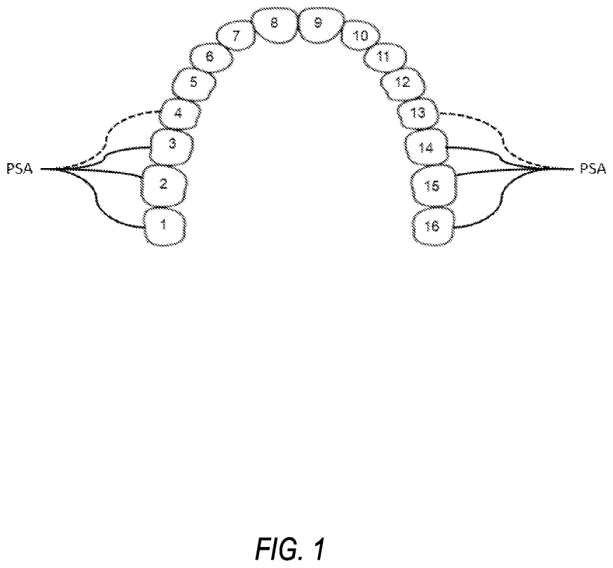

FIG. 1 provides an illustration summarizing the universal numbering maxillary teeth and shows that the molars (1-3 and 14-16) are enervated by the posterior superior alveolar as indicated by the solid line. In about one third of patients, the second premolar (4 and 13) are also enervated by the PSA, as indicated by the dashed line. For this reason, compositions such as Kovanaze, which are effective only on teeth of the maxillary arch that are innervated by the anterior superior alveolar (ASA) and middle superior alveolar (MSA) nerves will not be effective on patients where the second premolar is enervated by the PSA. In contrast, various compositions of the present disclosure are effective on the molars (teeth 1-3 and 14-16) as well as pre-molars that are enervated by the PSA (teeth 4 and 13 in about ⅓ of patients).

U.S. Pat. No. 8,580,282 B2 describes use of tetracaine in a non-injected formulation together with a vasoconstrictor and a pharmaceutically acceptable carrier, but explains that formulations comprising 4% (w/v) or more tetracaine were not as effective as 3% (w/v) tetracaine for inducing pulpal anesthesia. The examples in U.S. Pat. No. 8,580,282 describe topical anesthetization of several teeth, but do not describe complete anesthesia of the maxillary arch. Instead, the examples in U.S. Pat. No. 8,580,282 describe testing for anesthesia at the following soft tissue areas: distal to the apex of the tooth in the position of the maxillary first premolar at the deepest point in the buccal vestibule; apical to the maxillary lateral incisor at the deepest point in the labial vestibule, incisive papilla; at the confluence of the alveolar process and hard palate medial to the maxillary second premolar (near the greater palatine foramen), and only testing certain teeth that omit at least molars 1-2 and 15-16. KOVANAZE™ is a nasal spray containing tetracaine HCl and oxymetazoline HCl, but it is only indicated for regional anesthesia when performing a restorative procedure on teeth 4-13 and A-J in adults and children who weigh 40 kg or more. See, FDA Labelling for Kovanaze Nasay Spray. The dosage is 6 mg tetracaine HCL and 0.1 mg oxymetazoline HCl. Such formulations including only tetracaine and oxymetazoline as active agents/can result in undesirable side-effects in patients, including respiratory system disorders (81%), rhinorrhea (52%), nasal congestion (32%), nasal discomfort (26%), oropharyngeal pain (sore throat) (14%), intranasal hypoesthesia (10%), pharyngeal hypoesthesia (numb throat) (10%), throat irritation (9%), rhinalgia (6%), sneezing (4%), epistaxis (2%), nasal dryness (2%), nervous system disorders (22%), headache (10%), dysgeusia (8%), sinus headache (3%), dizziness (3%),sensory disturbance (2%), eye disorders 17%), lacrimation increased (watery eye) (13%), gastrointestinal disorders (9%), and oral discomfort (2%), per the Kovanaze Nasay Spray labelling. In several aspects, the pharmaceutical composition, spray device, or methods, of the present disclosure achieve an improved side effective profile relative to a Kovanaze Nasay Spray (which is approximately 3% tetracaine HCl and 0.05-0.1% oxymetazoline HCl) or control formulation having only tetracaine and a vasoconstrictor.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

### EXAMPLES

The following examples are illustrative and are not intended to limit the scope or content of the disclosure in any way.

Test Compositions

| | Composition | Composition | Composition | Composition | | Composition | |
|---|---|---|---|---|---|---|---|
| | A | B | C | D | | E | |
| Tetracaine | 4% | 4% | 4% | 4% | | 4% | |
| Bupivacaine | 0.25% | 0.25% | 0.5% | 0.5% | | 0.5% | |
| Oxymetazoline | Included | — | — | — | | — | |
| Phenylephrine | — | Included | Included | Included | | Included | |
| Eucalyptol | — | — | Included | — | | — | |
| Menthol | — | — | Included | — | | — | |
| Camphor | — | — | Included | — | | — | |
| Methylcellulose | — | — | — | Included | | — | |
| Dosing | 1 × 250 μl | 1 × 250 μl | 1 × 100 μl | 100 | μl | 100 | μl |
| (Volume) | spray | spray | spray | amount | | amount | |
| Dosing | 10 mg | 10 mg | 4 mg | 4 | mg | 4 | mg |

| | | | | | |
|---|---|---|---|---|---|
| (Tetracaine) | | | | | |
| Format | Spray | Spray | Spray | Gel | Spray |

Each test composition was compounded using ingredients that are commercially available in USP grade from Fischer Scientific (Waltham, MA).

Comparative Compositions

| | Comparative 1¹ | Comparative 2¹ | Comparative 3² |
|---|---|---|---|
| Tetracaine | 3% | 4% | 3% |
| Bupivacaine | — | — | |
| Oxymetazoline | 0.05% | 0.05% | 0.05% |
| Phenylephrine | — | — | — |
| Eucalyptol | — | — | — |
| Menthol | — | — | — |
| Camphor | — | — | — |
| Methylcellulose | — | — | — |
| Dosing | 2 × 200 μl | 2 × 200 μl | 2-3 × 200 μl |
| (Volume) | sprays | sprays | sprays |
| Dosing | 12 mg | 16 mg | 12-18 mg |
| (Tetracaine) | | | |
| Format | Spray | Spray | Spray |

Each comparative composition corresponds to a composition described in U.S. Pat. No. 8,580,282 B2 or the FDA Labelling for Kovanaze Nasal Spray, commercially available from St. Renatus (Fort Collins, CO).

### Measuring Anesthesia

The extent of anesthesia in one or more teeth is measured by using an electric pulp tester (EPT) set to a maximum value of 80 on a scale of 0-80. In some examples, a ENDO-ICE™ refrigerant spray is used, in a manner which is equivalent to the EPT test. For soft tissue, a pressure sensitive mechanical probe can be used. Successful anesthesia can also be measured by performing the indicated medical procedure.

### Measuring Adverse Events

All adverse events lasting more than 2 minutes were measured, evaluated for severity, and monitored for a week. Patients were followed up at 7-8 days. Adverse events that were monitored included intranasal hypoesthesia, rhinalgia, sinusitis, epistaxis, congestion, and running nose.

### Example 1—Local Anesthesia Using Composition A (Intranasal Spray)

Ten human patients were treated with a single 250 microliter spray of Composition A. The composition was administered by using a MAD NASAL™ intranasal mucosal atomization device, which is a 3 ml luer lock syringe equipped with an atomizer instead of a needle so as to be configured with for intranasal spray administration.

The results, over 10 patients, were that 94% of teeth numbered 5-12 and 90% of teeth numbered 1-4 and 13-16 achieved profound anesthesia. The onset of anesthesia occurred within 30 minutes after administration and continued for at least 20 additional minutes, and the profound extent of anesthesia was determined based on a lack of sensation when the tooth was stimulated with Endo-ice™. The Endo-ice stimulation can be estimated to correspond to stimulation with an electric pulp tester set to 80 on a standard EPT scale of 0 to 80.

Approximately half of the patients complained of significant and prolonged adverse effects. These side effects were similar to those previously observed for Comparative Compositions 1-3, and included one or more of congestion, rhinalgia, pain, sinusitis, or epistaxis.

### Example 2—Local Anesthesia Using Composition B (Intranasal Spray)

Ten human patients were treated with a single 250 microliter spray of Composition A according to the same procedure described in Example 1.

The results, over 10 patients, for both anesthesia and adverse events were substantially similar to Comparative Compositions 1-3, and Example 1.

### Example 3—Local Anesthesia Using Composition C (Intranasal Spray)

Ten human patients were treated with a single 100 microliter spray of Composition C according to the same procedure described in Example 1.

The results, over 10 patients, were that approximately 100% of teeth numbered 1-16 achieved profound anesthesia. The onset of anesthesia occurred within 30 minutes after administration and continued for at least 20 additional minutes, and the profound extent of anesthesia was determined based on a lack of sensation when the tooth was stimulated with Endo-ice™. The Endo-ice stimulation can be estimated to correspond to stimulation with an electric pulp tester set to 80 on a standard EPT scale of 0 to 80.

This result represents a significant improvement compared to Comparative Compositions 1-3, and Compositions A-B, in terms of efficacy and adverse events profile. Surprisingly, the including of eucalyptol, menthol, and camphor resulted in drastically reduced adverse events. For example, Comparative Compositions 1-3 and Examples 1-2 involved several significant side events, including medium-term adverse events that continued for 7 days. In contrast, Composition C resulted in minimal side effects, all of which were slight and went away within 4 hours.

### Example 4—Local Anesthesia Using Composition D (Individual Tooth)

A human patient was treated with Composition D, which is in the form of a gel, according to a modified procedure described in Example 1. The syringe was modified to have a syringe tip suitable for topical deliver to the gums instead of intranasally. A small amount corresponding to approximately 100-200 microliters of Composition D was applied at the top of the gum next to the top of a tooth and the composition was rubbed into the gums. Pulpal anesthesia was achieved without of adverse events.

### Example 5—Local Anesthesia Using Composition D (Maxillary Region)

A human patient was treated with Composition D, which is in the form of a gel, according to a modified procedure described in Example 1. The syringe was modified to have a syringe tip suitable for topical deliver to the infraorbital foramen of the patient instead of intranasally. A small amount corresponding to approximately 100-200 microliters of Composition D was applied at the infraorbital foramen of the patient. The maxillary region, including all maxillary teeth, achieved pulpal anesthesia without of adverse events.

### Example 6—Local Anesthesia Using Composition E (Wound Spray)

A human patient is treated with a single 100 microliter spray of Composition E according to a modified procedure described in Example 1. Composition E is configured for spraying onto the skin of a patient, rather than intranasally, and it is sprayed directly onto a suture site on skin of a patient. Sufficient anesthesia is achieved to provide or remove sutures without adverse events.

### Example 7—Needle-Free Local Anesthesia for Maxillary Palatal Injections

A delivery unit syringe of Composition D is opened and a Luer Lok syringe tip designed for delivery into the dental sulcus is added to the syringe. The tip of the applicator is placed in the dental sulcus of the maxillary tooth closest to the desired injection site and a small amount of Composition D is deposited. Immediately after a small amount of Composition D is then deposited on the palate at the actual injection site. A six-minute wait is observed, and at least pulpal anesthesia is achieved by use of Composition D. Injection is subsequently performed without pain to the patient.

### Example 8—Needle-Free Local Anesthesia for Periodontal Deep Scaling and Root Planing

A delivery unit syringe of Composition D is opened and a Luer Lok syringe tip designed for delivery into the dental sulcus is added to the syringe. The tip of the applicator is placed in the dental sulcus of the maxillary tooth or teeth that will be affected or treated for SRP and a small amount of Composition D is deposited. Immediately after, a small amount of Composition D is then deposited circumferentially around each tooth to be treated. A six-minute wait is then observed, and profound anesthesia is achieved. Treatment is subsequently performed without pain to the patient.

### Example 9—Needle-Free Local Anesthesia for Tissue Biopsy

A delivery unit syringe of Composition D is opened and a Luer Lok syringe tip designed for brush-on delivery is added to the syringe. The tip of the applicator is placed at the site of the biopsy. A small amount of Composition D is deposited over the area to be excised and around the margins to be removed. A six-minute wait is then observed, and profound anesthesia is achieved by use of Composition D, and excision is subsequently performed without pain to the patient.

### Example 10—Needle-Free Local Anesthesia for Frenectomy

A delivery unit syringe of Composition D is opened and a Luer Lok syringe tip is selected by the clinician for the purpose of delivery to the frenum to be treated. A small amount of Composition D is applied to the frenum. A second volume of Composition D from the syringe is deposited onto a small square of gauze and the wetted gauze is placed covering all tissue of the frenum to be excised and held in place with light pressure for six minutes. Profound anesthesia is achieved by use of Composition D, and frenectomy is subsequently performed without pain to the patient.

### Example 11—Needle-Free Local Anesthesia for Maxillary Crown and Bridge Procedures Involving Retraction

A delivery unit syringe of Composition D is opened and a Luer Lok syringe tip designed for delivery into the dental sulcus is added to the syringe. The tip of the applicator is placed in the dental sulcus of the maxillary tooth or teeth that will be affected or treated for retraction and cord placement prior to impressions and temporization, and a small amount of Composition D is deposited. Immediately after a small amount of Composition D is then deposited on the retraction cord itself and circumferentially around each tooth to be treated. A six-minute wait is then observed before treatment is initiated. The result is that profound anesthesia is achieved by use of Composition D, and treatment is subsequently performed without pain to the patient.

### Example 12—Needle-Free Local Anesthesia for Suture Removal

A delivery unit syringe of Composition D is opened and a Luer Lok syringe tip designed for brush-on delivery is added to the syringe. The tip of the applicator is placed at the site of the sutures to be removed/manipulated and a small amount of Composition D is applied to the site. A six-minute wait is then observed before sutures are removed. The result is that profound anesthesia is achieved by use of Composition D, and sutures are subsequently removed without pain to the patient.

### Example 13—Needle-Free Local Anesthesia for Implant Uncovering or Tissue Punch

A delivery unit syringe of Composition D is opened and a Luer Lok syringe tip designed for brush-on delivery is added to the syringe. The tip of the applicator is placed at the site of the implant uncovering or tissue punch. A small amount of Composition D is deposited over the area to be excised and around the margins of the manipulation. A six-minute wait is then observed prior to excision or tissue punch. The result is that profound anesthesia is achieved by use of Composition D, and the procedure is subsequently performed without pain to the patient.

TABLE 3
Comparison Results

| | Comparative | Comparative | Comparative | Composition | Composition | Composition |
|---|---|---|---|---|---|---|
| | 1¹ | 2¹ | 3² | A | B | C |
| Tetracaine | 3% | 4% | 3% | 4% | 4% | 4% |
| Bupivacaine | — | — | — | 0.25% | 0.25% | 0.5% |
| Oxymetazoline | 0.05% | 0.05% | 0.05% | Included | — | — |
| Phenylephrine | — | — | — | — | Included | Included |
| Eucalyptol | — | — | — | — | — | Included |
| Menthol | — | — | — | — | — | Included |
| Camphor | — | — | — | — | — | Included |
| Methylcellulose | — | — | — | — | — | Included |
| Dosing | 2 × 200 μl | 2 × 200 μl | 2-3 × 200 μl | 1 × 250 μl | 1 × 250 μl | 1 × 100 μl |
| (Volume) | sprays | sprays | sprays | spray | spray | spray |
| Dosing | 12 mg | 16 mg | 12-18 mg | 10 mg | 10 mg | 4 mg |
| (Tetracaine) | | | | | | |
| Format | Spray | Spray | Spray | Spray | Spray | Spray |
| Adverse Effects | Approx. | Approx. | Approx. | Approx. | Approx. | Virtually |
| | Half of | Half of | Half of | Half of | Half of | eliminated |
| | Patients | Patients | Patients | Patients | Patients | |
| Anesthesia³ | — | — | ~96% of | ~94% of | ~94% of | ~100% |
| Result | | | teeth² | teeth | teeth | |
| (Teeth 5-12) | | | | | | |
| Anesthesia³ | — | — | ~63% of | ~90% of | ~90% of | ~100% |
| Result | | | teeth² | teeth | teeth | |
| (Teeth 4, 13) | | | | | | |
| Anesthesia³ | — | — | N/A² | ~90% of | ~90% of | ~100% |
| Result | | | | teeth | teeth | |
| (Teeth 1-3 | | | | | | |
| and 14-16) | | | | | | |
| Anesthesia³ | 60% of | 22% of | — | — | — | ~100% |
| Result (All | test | test | | | | |
| Teeth). | teeth¹ | teeth¹ | | | | |

¹Data corresponding to data from U.S. Pat. No. 8,580,282 B2.
²Data corresponding to data from clinical studies described in FDA Kovanaze Labelling.
³Composition percentages are in units of (w/v). Anesthesia corresponds to maximum EPT stimulation of 80 on a scale of 0-80.

### DISCUSSION

Each of the tested combination formulations resulted in similar or greater efficacy compared to the Comparative Examples, but surprisingly did not result in significantly greater adverse reactions despite involving a higher concentration of tetracaine and an additional therapeutic agent, bupivacaine. It is also surprising that equal or greater efficacy was achieved by using less material overall, and by use of only a single spray. See dosing (volume) and dosing (tetracaine) in Table 3. Moreover, this result is further surprising because the disclosure of U.S. Pat. No. 8,580,282 B2, discussed hereinabove, reports that 4% tetracaine is less effective than 3% and that bupivacaine was not as effective as tetracaine. See, cols. 7, 13, and 14 of U.S. Pat. No. 8,580,282 B2.

### Exemplary Embodiments

The following exemplary embodiments are provided, the numbering of which is not to be construed as designating levels of importance.
Embodiment 1 provides a pharmaceutical composition comprising: 3.6% (w/v) or more tetracaine, or a pha15rmaceutically acceptable salt thereof; 0.1% (w/v) or more bupivacaine, or a pharmaceutically acceptable salt thereof; a vasoconstrictor; and a pharmaceutically acceptable carrier.
Embodiment 2 provides the pharmaceutical composition of Embodiment 1, comprising about 4.0% (w/v) or more tetracaine, or a pharmaceutically acceptable salt thereof.
Embodiment 3 provides the pharmaceutical composition of Embodiment 1 or 2, comprising about 0.5% (w/v) or more bupivacaine, or a pharmaceutically acceptable salt thereof.
Embodiment 4 provides the pharmaceutical composition of any one of Embodiments 1-3, wherein the vasoconstrictor is 0.01-1.0% (w/v) of the composition.
Embodiment 5 provides the pharmaceutical composition of any one of Embodiments 1-4, wherein the vasoconstrictor is phenylephrine, or a pharmaceutically acceptable salt thereof.
Embodiment 6 provides the pharmaceutical composition of any one of Embodiments 1-4, comprising: about 4.0-6.0% (w/v) tetracaine, or a pharmaceutically acceptable salt thereof; about 0.25-1.0% (w/v) bupivacaine, or a pharmaceutically acceptable salt thereof; about 0.01-1.0% (w/v) phenylephrine; and a pharmaceutically acceptable carrier.
Embodiment 7 provides the pharmaceutical composition of any one of Embodiments 1-6, comprising: about 4.0% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier.
Embodiment 8 provides the pharmaceutical composition of any one of Embodiments 1-7, which is a topical composition.
Embodiment 9 provides the pharmaceutical composition of any one of Embodiments 1-8, which is an aqueous composition.
Embodiment 10 provides the pharmaceutical composition of any one of Embodiments 1-9, which is a sprayable dosage form, or an aerosol.
Embodiment 11 provides the pharmaceutical composition of any one of Embodiments 1-10, comprising eucalyptol, menthol, camphor, or any combination thereof.
Embodiment 12 provides the pharmaceutical composition of any one of Embodiments 1-11, comprising a thickening agent.
Embodiment 13 provides the pharmaceutical composition of Embodiment 12, wherein the thickening agent is methylcellulose.
Embodiment 14 provides the pharmaceutical composition of any one of Embodiments 1-13, having a viscosity of about 3000-6000 cP at 25° C.
Embodiment 15 provides a spray device, comprising the pharmaceutical composition of any one of Embodiments 1-13, a reservoir, and an atomizer.
Embodiment 16 provides the spray device of Embodiment 15, wherein the atomizer is configured to deliver an aerosolized dose of less than 200 microliters of the pharmaceutical composition.
Embodiment 17 provides the spray device of Embodiment 15, wherein the atomizer is configured to deliver a single aerosolized dose of about 100 microliters of the pharmaceutical composition.
Embodiment 18 provides the spray device of any one of Embodiments 15-17, wherein the atomizer is configured to deliver an aerosolized dose about 2-5 mg of tetracaine.
Embodiment 19 provides the spray device of any one of Embodiments 15-18, which is configured as an intranasal spray for delivering the aerosol to nasal and oropharyngeal mucous membranes.
Embodiment 20 provides the spray device of any one of Embodiments 15-19, comprising a syringe, which provides the reservoir and drives the pharmaceutical composition through the atomizer.
Embodiment 21 provides a method of providing local anesthesia of a site of a patient, comprising topically administering to the patient the pharmaceutical composition of any one of Embodiments 1-14, or the spray device of any one of Embodiments 15-20.
Embodiment 22 provides the method of Embodiment 21, wherein the site comprises a tooth or an oral tissue.
Embodiment 23 provides the method of Embodiment 21 or 22, wherein the site comprises at least one adult tooth numbered 1-3 or 13-16, or adult tooth numbered 4 or 12 that is enervated by a posterior superior alveolar (PSA) nerve in the patient, based on universal tooth numbering.
Embodiment 24 provides the method of any one of Embodiments 21-23, wherein the site is an individual maxillary tooth.
Embodiment 25 provides the method of any one of Embodiments 21-24, wherein the pharmaceutical composition is topically administered by placing it at the patient's upper gum near a root of a maxillary tooth for which anesthesia is desired.
Embodiment 26 provides the method of any one of Embodiments 21-23, wherein the site is a maxillary region of teeth, tissue, or both.
Embodiment 27 provides the method of Embodiment 26, wherein the maxillary region is all maxillary teeth, two or more maxillary molars, all maxillary molars, all teeth in an upper right quadrant, all teeth in an upper left quadrant, a hard palate, a soft palate, gums, an implant, a posterior superior alveolar (PSA) nerve, or any combination thereof.
Embodiment 28 provides the method of any one of Embodiments 21-24 or 26-27, wherein the pharmaceutical composition is topically administered by placing it at the patient's infraorbital foramen.
Embodiment 29 provides the method of any one of Embodiments 21-24 or 26-27, wherein the pharmaceutical composition is administered intranasally as a spray, which is delivered ipsilaterally to the site for which anesthesia is desired.
Embodiment 30 provides the method of Embodiment 29, wherein the pharmaceutical composition comprises eucalyptol, menthol, camphor, or any combination thereof.
Embodiment 31 provides the method of Embodiment 30, wherein the patient is free from adverse events of congestion, rhinalgia, sinusitis, and epistaxis for 7 days following administration of the pharmaceutical composition.
Embodiment 32 provides the method of Embodiment 21, wherein the site is a non-oral mucosal tissue, an epidermal tissue, a dermal tissue, or a sub-dermal tissue.
Embodiment 33 provides the method of Embodiment 21-24 or 26-27, wherein the site is an open wound, a laceration, an implant site, a biopsy site, or a suture site.
Embodiment 34 provides the method of any one of Embodiments 21-24, 26-27 or 29-33, wherein the pharmaceutical composition is administered in the form of a topical spray delivered directly to the site for which anesthesia is desired.
Embodiment 35 provides the method of any one of Embodiments 21-24, 26-27, or 29-34, wherein the pharmaceutical composition is administered in an aerosolized dose of less than 200 microliters of the pharmaceutical composition.
Embodiment 36 provides the method of Embodiment 35, wherein the pharmaceutical composition is administered in a single aerosolized dose of about 100 microliters of the pharmaceutical composition.
Embodiment 37 provides the method of any one of Embodiments 21-36, wherein the pharmaceutical composition is administered at dose of about 2-5 mg of tetracaine.
Embodiment 38 provides the method of Embodiment 37, wherein the pharmaceutical composition is administered at dose of about 4 mg of tetracaine.
Embodiment 39 provides the method of any one of Embodiments 21-38, which results in anesthesia corresponding to a lack of sensation at the site when it is stimulated with an electric pulp tester (EPT) set to at least 65 on a standard EPT scale of 0 to 80.
Embodiment 40 provides the method of any one of Embodiments 21-29, which results in anesthesia within 30 minutes after administration and continuing for at least 20 additional minutes, and wherein the anesthesia corresponds to a lack of sensation at the site when it is stimulated with an electric pulp tester set to 80 on a standard EPT scale of 0 to 80.
Embodiment 41 provides the method of Embodiment 21-40, which achieves anesthesia free of any injection.
Embodiment 42 provides the method of any one of Embodiments 21-41, wherein the local anesthesia is suitable for performing a restorative procedure in a tooth of an adult or a child that weighs 40 kg or more.
Embodiment 43 provides the method of any one of Embodiments 21-41, wherein the local anesthesia is suitable for performing one or more of an axillary palatal injection, deep scaling, root planning, tissue biopsy, frenectomy, crown or bridge procedure involving retraction, suture placement, or an implant uncovering procedure.
Embodiment 44 provides the method of any one of Embodiments 21-43, further comprising performing a medical procedure selected from one or more of an axillary palatal injection, deep scaling, root planning, tissue biopsy, frenectomy, crown or bridge procedure involving retraction, suture placement, an implant uncovering procedure, or a restorative procedure in a tooth numbered 1-3 or 13-16, based on universal tooth numbering, or anesthesia of adult tooth numbered 4 or 12 that is enervated by a posterior superior alveolar (PSA) nerve
Embodiment 45 provides a method of treating a patient without injecting a local anesthetic, comprising topically administering the pharmaceutical composition of any one of Embodiments 1-14, or applying the spray device of any one of Embodiments 15-20, to a patient in need thereof; and performing a medical procedure selected from one or more of an axillary palatal injection, deep scaling, root planning, tissue biopsy, frenectomy, crown or bridge procedure involving retraction, suture placement, an implant uncovering procedure, or a restorative procedure in a tooth numbered 1-3 or 13-16, based on universal tooth numbering, or anesthesia of adult tooth numbered 4 or 12 that is enervated by a posterior superior alveolar (PSA) nerve.
Embodiment 46 provides a topical gel composition, comprising: about 4% (w/v) tetracaine, or a pharmaceutically acceptable salt thereof; about 0.5% (w/v) bupivacaine, or a pharmaceutically acceptable salt thereof; phenylephrine; a pharmaceutically acceptable carrier; and a thickening agent at amount sufficient to provide a viscosity of about 4000-5000 cP at 25° C.
Embodiment 47 provides a sprayable composition, comprising: about 4% (w/v) tetracaine, or a pharmaceutically acceptable salt thereof; about 0.5% (w/v) bupivacaine, or a pharmaceutically acceptable salt thereof; phenylephrine; a pharmaceutically acceptable carrier; and eucalyptol, menthol, and camphor.
Embodiment 48 provides a spray device, comprising an aqueous pharmaceutical composition, a reservoir, and an atomizer, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier.
Embodiment 49 provides a method of providing local anesthesia to an adult molar numbered 1-3 or 13-16, based on universal tooth numbering, comprising topically administering a pharmaceutical composition to a tissue proximate to the molar being treated in a patient in need thereof, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier.
Embodiment 50 provides a method of providing local anesthesia to a tissue site of a patient, comprising topically administering a pharmaceutical composition directly to the tissue of the patient in need thereof, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier, wherein the tissue site is an oral tissue, a non-oral mucosal tissue, an epidermal tissue, a dermal tissue, a sub-dermal tissue, an open wound, a laceration, an implant site, a biopsy site, or a suture site.
Embodiment 51 provides a method of providing local anesthesia to a tissue site of a patient, comprising topically administering a pharmaceutical composition directly to the tissue of the patient in need thereof, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier, wherein the tissue site is an oral tissue, a non-oral mucosal tissue, an epidermal tissue, a dermal tissue, a sub-dermal tissue, an open wound, a laceration, an implant site, a biopsy site, or a suture site.
Embodiment 52 provides a composition comprising: about 4.0% (w/v) tetracaine HCl; about 0.5% (w/v) bupivacaine HCl; about 1.0% (w/v) phenylephrine HCl; about 0.06% (w/v) sodium hydroxide 0.1N; and sterile water.
Embodiment 53 provides a composition comprising: about 4.0% (w/v) tetracaine HCl; about 0.5% (w/v) bupivacaine HCl; about 1.0% (w/v) phenylephrine HCl; about 0.5% (w/v) stevia; about 0.5% (w/v) acesulfame potassium; about 0.06% (w/v) sodium hydroxide 0.1N; about 0.9% (w/v) benzyl alcohol; about 2.0% (w/v) hydroxyethyl cellulose; and sterile water.
Embodiment 54 provides a composition substantially consisting of: about 4.0% (w/v) tetracaine HCl; about 0.5% (w/v) bupivacaine HCl; about 1.0% (w/v) phenylephrine HCl; about 0.06% (w/v) sodium hydroxide 0.1N; and sterile water.
Embodiment 55 provides a composition substantially consisting of: about 4.0% (w/v) tetracaine HCl; about 0.5% (w/v) bupivacaine HCl; about 1.0% (w/v) phenylephrine HCl; sodium hydroxide 0.1N; about 2.0% (w/v) hydroxyethyl cellulose; and sterile water.
Embodiment 56 provides a composition consisting of: about 4.0% (w/v) tetracaine HCl; about 0.5% (w/v) bupivacaine HCl; about 1.0% (w/v) phenylephrine HCl; about 0.5% (w/v) stevia; about 0.5% (w/v) acesulfame potassium; about 0.06% (w/v) sodium hydroxide 0.1N; about 0.9% (w/v) benzyl alcohol; about 2.0% (w/v) hydroxyethyl cellulose; and sterile water.
Embodiment 57 provides a method of providing local anesthesia to a tissue site of a patient, comprising topically administering a pharmaceutical composition directly to the tissue of the patient in need thereof, wherein the pharmaceutical composition comprises: about 4% (w/v) tetracaine, or a pharmaceutically acceptable salt thereof; about 0.5% (w/v) bupivacaine, or a pharmaceutically acceptable salt thereof; phenylephrine; and a pharmaceutically acceptable carrier, wherein the tissue site is an oral tissue, a non-oral mucosal tissue, an epidermal tissue, a dermal tissue, a sub-dermal tissue, an open wound, a laceration, an implant site, a biopsy site, or a suture site.
Embodiment 58 provides a method of providing local anesthesia to a tissue site of a patient, wherein a composition of any of the proceeding Embodiments is administered in the form of a topical spray delivered directly to the site for which anesthesia is desired, and the site is an a non-oral mucosal tissue, a sub-dermal tissue, an open wound, or a laceration.
Embodiment 59 provides a method of providing local anesthesia to a tissue site of a patient, wherein a composition of any of the proceeding Embodiments is administered to the patient without any injection or any pain.
Embodiment 60 provides a method of providing local anesthesia to a tissue site of a patient, wherein a composition of any of the proceeding Embodiments is delivered to the tissue site of the patient at a dosage of 1 ul to 500 ul.
Embodiment 61 provides a method of providing local anesthesia to a tissue site of a patient, wherein a composition of any of the proceeding Embodiments is delivered to the tissue site of the patient via a metered dose of about 100 ul.
Embodiment 62 provides the use of any one of the preceding compositions for manufacturing a medicament.
Embodiment 63 provides the use of any one of the preceding compositions for manufacturing a local anesthetic.
Embodiment 64 provides any one of the preceding compositions for use in providing local anesthesia.
Embodiment 65 provides any one of the preceding compositions for use in a method providing local anesthesia topically without injection.
Embodiment 66 provides any one of the preceding compositions for use in any of the preceding methods.
Embodiment 67 provides a composition, spray device, or method of any one or any combination of Embodiments 1-66 optionally configured such that each element or option recited are available to use or select from.

What is claimed is:

1. A pharmaceutical composition comprising:
a) 3.6% (w/v) or more tetracaine, or a pharmaceutically acceptable salt thereof;
b) 0.1% (w/v) or more bupivacaine, or a pharmaceutically acceptable salt thereof;
c) a vasoconstrictor; and
d) a pharmaceutically acceptable carrier.

2. The pharmaceutical composition of claim 1, comprising:
a) about 4.0% (w/v) tetracaine, or a pharmaceutically acceptable salt thereof;
b) about 0.5% (w/v) bupivacaine, or a pharmaceutically acceptable salt thereof;
c) about 1% (w/v) phenylephrine; and
d) a pharmaceutically acceptable carrier.

3. The pharmaceutical composition of claim 1, which is a topical composition.

4. The pharmaceutical composition of claim 1, which is a sprayable dosage form, or an aerosol, and optionally comprises eucalyptol, menthol, camphor, or any combination thereof.

5. The pharmaceutical composition of claim 1, which is a topical composition comprises a thickening agent, a viscosity of about 3000-6000 cP at 25° C., or both.

6. A spray device comprising the pharmaceutical composition of claim 1, a reservoir, and an atomizer.

7. The spray device of claim 6, wherein the atomizer is configured to deliver an aerosolized dose of less than 200 microliters of the pharmaceutical composition.

8. The spray device of claim 6, wherein the atomizer is configured to deliver a single aerosolized dose of about 100 microliters of the pharmaceutical composition.

9. The spray device of claim 6, wherein the atomizer is configured to deliver an aerosolized dose about 2-5 mg of tetracaine.

10. A method of providing local anesthesia of a site of a patient, comprising topically administering to the patient the pharmaceutical composition of claim 1.

11. The method of claim 10, wherein the site comprises at least one adult tooth numbered 1-3 or 13-16, or adult tooth numbered 4 or 12 that is enervated by a posterior superior alveolar (PSA) nerve in the patient, based on universal tooth numbering.

12. The method of claim 10, wherein the pharmaceutical composition is topically administered by placing it at the patient's upper gum near a root of a maxillary tooth for which anesthesia is desired.

13. The method of claim 10, wherein the pharmaceutical composition is topically administered by placing it at the patient's infraorbital foramen.

14. The method of claim 10, wherein the pharmaceutical composition is administered intranasally as a spray, which is delivered ipsilaterally to the site for which anesthesia is desired, and the intranasal spray comprises eucalyptol, menthol, camphor, or any combination thereof.

15. The method of claim 10, wherein the pharmaceutical composition is administered in the form of a topical spray delivered directly to the site for which anesthesia is desired, and the site is an oral tissue, a non-oral mucosal tissue, an epidermal tissue, a dermal tissue, a sub-dermal tissue, an open wound, a laceration, an implant site, a biopsy site, or a suture site.

16. The method of claim 10, wherein the pharmaceutical composition is administered in an aerosolized dose of less than 200 microliters of the pharmaceutical composition.

17. The method of claim 10, wherein the pharmaceutical composition is administered in a single aerosolized dose of about 100 microliters of the pharmaceutical composition.

18. The method of claim 10, wherein the pharmaceutical composition is administered at dose of about 2-5 mg of tetracaine.

19. The method of claim 10, which results in anesthesia corresponding to a lack of sensation at the site when it is stimulated with an electric pulp tester (EPT) set to at least 65 on a standard EPT scale of 0 to 80.

20. The method of claim 10, which results in anesthesia within 30 minutes after administration and continuing for at least 20 additional minutes, and wherein the anesthesia corresponds to a lack of sensation at the site when it is stimulated with an electric pulp tester set to 80 on a standard EPT scale of 0 to 80.
